(19) United States
(12) Patent Application Publication
(10) Pub. No.: US 2018/0037726 A1
(43) Pub. Date: Feb. 8, 2018

(54) ULTRA HIGHMOLECULAR WEIGHT POLYETHYLENE COMPOSITIONS

(71) Applicant: Veerag Mehta, South Plainfield, NJ (US)

(72) Inventor: Veerag Mehta, South Plainfield, NJ (US)

(21) Appl. No.: 15/642,701

(22) Filed: Jul. 6, 2017

Related U.S. Application Data
(60) Provisional application No. 62/370,740, filed on Aug. 4, 2016.

(51) Int. Cl. C08L 23/06; A61L 31/04; A23L 29/00; D01F 6/46; D01F 6/94; C08J 5/18; C08L 83/04

(52) U.S. Cl. CPC C08L 23/06; C08J 5/18; A61L 31/041; C08L 83/04; D01F 6/46; D01F 6/94; A23L 29/00; C08L 2203/16; C08L 2203/18; C08J 2323/06; C08J 2383/04; C08J 2483/04; C08L 2205/06; A23V 2002/00

(57) ABSTRACT

Ultrahigh molecular weight, or high molecular weight silicone is added to ultrahigh molecular weight polyethylene polymer during the compounding process. It creates a material composition that is processable by traditional injection molding and extrusion processes that rely on shear as a primary means of melting and helping to shape end products.

This application is a utility application from U.S. provisional patent application Ser. No. 62/370,740, filed Aug. 4, 2016 from which priority is claimed.

### BACKGROUND OF THE INVENTION

It Is been well known that ultrahigh molecular weight Polyethylene (UHMWPE) has excellent wear resistant properties, however it has been limited on processing due to its inability to be processed by traditional processing means like injection molding and extrusion.

The use of ultrahigh molecular weight siloxane during the compounding of UHMWPE changes the processability of UHMWPE so that the resin can be processed by traditional processes like injection molding and extrusion. In addition the wear resistance of the material is further enhanced over standard UHMWPE.

It is well known that ultrahigh molecular weight polyethylene, which is commercially available as a powder, can only be converted by processes like ram extrusion or compression molding. This has limited the use and applicability of the polymer. The reason for the limitation is because of the extremely high viscosity of the UHMWPE such that it has difficulty melting and flowing while going through traditional processes like injection molding that rely heavily on shear as a means of melting and fusing the pellets or powder together.

### THE INVENTION

Recently it has been discovered that if ultrahigh molecular weight, or high molecular weight silicone is added to UHMWPE during the compounding process, it creates a material composition that is processable by traditional injection molding and extrusion processes that rely on shear as a primary means of melting and helping to shape end products.

Thus, what is disclosed and claimed herein is, in one embodiment, a composition of matter comprising 50 to 99 weight percent ultrahigh molecular weight polyethylene having a minimum molecular weight of at least 3×10⁶ g/mole and 1 to 50 weight percent of ultrahigh molecular weight silicone polymer having a viscosity of greater than 500,000 cst at room temperature, all based on the total weight of A. and B.

In a second embodiment, there is disclosed and claimed a composition of matter comprising 50 to 99 weight percent ultrahigh molecular weight polyethylene having a minimum molecular weight of at least 3×10⁶ g/mole and 1 to 50 weight percent of high molecular weight silicone polymer having viscosity of greater than 60,000 cst at room temperature, all based on the total weight of A. and B.

### DETAILED DESCRIPTION OF THE INVENTION

In the first embodiment, the composition of matter comprises 50 to 99 weight percent of ultrahigh molecular weight polyethylene having a minimum molecular weight of at least 3×16⁶ g/mole and 1 to 50 weight percent of ultrahigh molecular weight silicone polymer having a viscosity of greater than 500,000 cst at room temperature. All of the percentages are based cm the total weight of the ultrahigh molecular weight polyethylene and tne ultrahigh molecular weight silicone polymer.

In the second embodiment of the invention, the composition of matter comprises 50 to 99 weight percent ultrahigh molecular weight polyethylene having a minimum molecular weight of at least 3×10⁶ g/mole and 1 to 50 weight percent of high molecular weight silicone polymer. Again, the percentages are based on the total weight of the ultrahigh molecular weight polyethylene and the ultrahigh molecular weight silicone polymer.

In each case of the silicone polymer, the silicone can have functional groups on them, including but not limited to hydroxyl, vinyl, phenyl, and fluoro which can be present as end groups or pendent to the molecule.

Contemplated within the scope of this invention is the use of a second lubricant (in addition to the silicone polymer) in the form of a particle, the particle being selected from the group consisting of polytetrafluoroethylene, boron nitride, aramid fiber, nolybdenum disulfide, graphite, and carbon fiber. It has been found that stabilizers and colorants can be added to the composition without deleterious effects.

It has been discovered that the compositions can be extruded into fibers, films, sheets, and tubes, among other forms. These forms can be used in medical devices, food processing, and as pharmaceutical processing components.

What is claimed is:

1. A composition of matter comprising:
(i) 50 to 99 weight percent ultrahigh molecular weight polyethylene having a minimum molecular weight of at least 3×10⁶ g/mole and
(ii) 1 to 50 weight percent of ultrahigh molecular weight silicone polymer having a viscosity of greater than 500,000 cst at room temperature, all based on the total weight of A. and B.

2. A composition as claimed in claim 1 wherein there is also present a stabilizer.

3. A composition as claimed in claim 1 wherein there is also present a colorant.

4. A composition as claimed in claim 1 wherein there is also present a second lubricant in the form of a particle, said particle selected from the group consisting of polytetrafluoroethylene, boron nitride, aramid fiber, molybdenum disulfide, graphite, and carbon fiber.

5. A composition of matter comprising:
(i) 50 to 99 weight percent ultrahigh molecular weight polyethylene having a minimum molecular weight of at least 3×10⁶ g/mole and
(ii) 1 to 50 weight percent of high molecular weight silicone poiynier having viscosity of greater than 60,000 cst at room temperature, all based on the total weight of A. and B.

6. A composition as claimed in claim 5 wherein there is also present a stabilizer.

7. A composition as claimed in claim 5 wherein there is also present a colorant.

6. Extruded fibers extruded from the composition of claim 1

8. Extruded, films extruded from the composition of claim 1.

9. Extruded sheets extruded from the composition of claim 1.

10. Extruded tubes extruded from the composition of claim 1.

11. Medical devices extruded from the composition of claim 1.

12. Food processing components manufactured from the composition of claim 1.

13. Pharmaceutical process components extruded from the composition of claim 1.

14. Injection molded parts manufactured from the composition of claim 1.

15. Extruded fibers extruded from the composition of claim 5.

16. Extruded films extruded from the composition of claim 5.

17. Extruded sheets extruded from the composition of claim 5.

18. Extruded tubes extruded from the composition of claim 5.

19. Medical devices extruded from the composition of claim 5.

20. Food processing components extruded from the composition of claim 5.

22. Pharmaceutical process components extruded from the composition of claim 5.

23. Injection molded parts extruded from the composition of claim 5.
